(19) United States
(12) Patent Application Publication
(10) Pub. No.: US 2008/0032321 A1
(43) Pub. Date: Feb. 7, 2008

(54) SYSTEM AND METHODS FOR ANALYZING IMAGES OF TISSUE SAMPLES

(75) Inventors: FIONA GINTY, SARATOGA SPRINGS, NY (US); ROBERT JOHN FILKINS, NISKAYUNA, NY (US); HARVEY ELLIS CLINE, SCHENECTADY, NY (US); MICHAEL CHRISTOPHER MONTALTO, ALBANY, NY (US); SUDESHNA ADAK, BANGALORE (IN); ALI CAN, TROY, NY (US); MICHAEL JOHN GERDES, ALBANY, NY (US); MELINDA LARSEN, ALBANY, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 11/686,649

(22) Filed: Mar. 15, 2007

Related U.S. Application Data
(63) Continuation-in-part of application No. 11/500,028, filed on Aug. 7, 2006.
Application No. 11/500,028 is a continuation-in-part of application No. 11/606,582, filed on Nov. 30, 2006.
Application No. 11/606,582 is a continuation-in-part of application No. 11/680,063, filed on Feb. 28, 2007.

(51) Int. Cl. G01N 33/53

(52) U.S. Cl. 435/15

(57) ABSTRACT

A system for analyzing tissue samples, comprising: a storage device for at least temporarily storing one or more images of one or more cells, wherein at least one of the images is indicative of one or more channels comprising a receptor tyrosine kinase (RTK); and a processing device that determines an extent to which one or more of the RTKs may have translocated from at least one subcellular region to another subcellular region of one or more of the cells; and generates a score based at least in part on the RTK translocation.

### CROSS REFERENCE TO RELATED APPLICATIONS

This application is a continuation-in-part of U.S. patent application Ser. No. 11/500,028, entitled “System and Method for Co-Registering Multi-Channel Images of a Tissue Micro Array”, filed on Aug. 7, 2006, which is herein incorporated by reference; a continuation-in-part of U.S. patent application Ser. No. 11/606,582, entitled “System and Methods for Scoring Images of a Tissue Micro Array, filed on Nov. 30, 2006, which is herein incorporated by reference; and a continuation-in-part of U.S. application Ser. No. 11/680,063, entitled Automated Segmentation of Image Structures, filed on Feb. 28, 2007, which is herein incorporated by reference.

### BACKGROUND

The invention relates generally to tissue processing, image analysis, and disease prognosis.

Cancer histopathology diagnosis has historically been based on cellular morphology using hematoxylin and eosin (H&E) stained biopsy tissue with bright field microscopy. Today, oncogenes are detected with immunohistochemistry (IHC) stains in selected diagnostic exams to prescribe drug therapy with new cancer drugs that are only effective in a fraction of patients (e.g. Her2 test and herceptin). Although IHC has been commonly used to measure protein expression, IHC has several limitations. IHC methods are not standardized, limiting comparability between labs and provide only subjective measurements of total target protein expression. IHC is also unable to simultaneously measure protein expression in more than one cellular compartment.

Immunohistochemistry using peroxidase enzyme amplification and diaminobenzidine (DAB) substrate is the standard technique used to assess the spatial location and qualitative expression patterns of proteins in tumor and other tissue samples. However, this technique is inadequate to finely assess the quantity of proteins in a tissue sample. This limitation is due, in part, to the low dynamic range of bright field microscopy, which is further exacerbated by the inherent enzymatic amplification of peroxidase-based immunostaining. Thus, at best, current quantification requires the subjective interpretation of low (1+), medium (2+) and high (3+) intensity. Although such interpretations have become standard in clinical and research practice, most assays are currently performed using non-standardized immunohistochemical assessment with subjective interpretation, raising concerns about quantitative accuracy. This also raises concerns about inappropriate therapeutic stratification and compromised clinical outcome.

As an example, receptor tyrosine kinases (RTK) possess an extracellular ligand binding domain, a transmembrane domain and an intracellular catalytic domain. The transmembrane domain anchors the receptor in the plasma membrane, while the extracellular domains bind growth factors. The intracellular kinase domains of RTKs can be divided into two classes: those containing a stretch of amino acids separating the kinase domain and those in which the kinase domain is continuous. Activation of the kinase is achieved by ligand binding to the extracellular domain, which induces dimerization of the receptors. Receptors thus activated are able to autophosphorylate tyrosine residues outside the catalytic domain via cross-phosphorylation. The results of this auto-phosphorylation are stabilization of the active receptor conformation and the creation of phosphotyrosine docking sites for proteins that transduce signals within the cell.

Simultaneous measurement of subcellular (including membrane, cytoplasm, nuclear) RTKs provides information on cell activation status and disease pathology. However, simultaneous quantification of subcellular RTK expression is currently not possible with available technologies. For example, total cMet expression may be estimated visually as the percent of cells staining positive and the intensity of staining for those cells. While this approach may be used to provide a categorical result ranging from 0 (no expression) to +3 (high expression) and provides some intrinsic value, this approach is limited to evaluating a single marker.

Automated quantitative image analysis of multiple proteins simultaneously in the same tissue section are desired as tools for more accurate, rapid diagnosis of cancer phenotype and directing patients to appropriate therapies. Although there exists certain techniques, such as DNA microarray technologies, that provide one approach for the simultaneous measurement of multiple disease-associated genes, there are concerns that these techniques may not indicate actual protein expression. These techniques also do not provide information on the cellular localization within the context of the tissue specimen. Other more recently developed methods for automated and quantitative in situ compartmental protein expression, such as AQUA™, which uses fluorescent-based immunohistochemistry, digital microscopy and post-image processing, have been used to demonstrate subcellular location of biomarkers in a fully quantitative manner. However, AQUA™ has an upper limit of five molecular markers that can be measured at one time in a single tissue section. However, these methods also do not provide an adequate means for assessing likely clinical outcomes.

### BRIEF DESCRIPTION

Translocation activities are used in the embodiments of the methods described as indicators of cancers, including but not limited to colon cancer and other epithelial-based cancers and inflammatory diseases. For example, the methods provide simultaneous, accurate measurement of membrane, cytoplasm, and/or nuclear levels of cMet RTK, which is used as a biomarker in one or more of the embodiments described for colon cancer. These methods also provide a superior approach to traditional IHC and new insights into cell activation status and disease pathology.

The methods and systems of the invention create an automated image analysis framework that can be used automatically quantify and score digital images of tissue samples for a variety of applications including, but not limited to, screening patients for cancer, and even specific stages and types of cancer, and other diseases and to identify and quantify multiple biomarkers in a single tissue sections to develop cancer drug therapies, toxicology evaluation and research. The technical effect of these methods and systems is to enable automatic analysis of multi-channel tissue images; simultaneous analysis/quantification of multiple biomarkers; high throughput analysis of large patient cohorts; spatially resolved quantification; and compartment based biomarker analysis for cancer scoring. Multiple biomarkers that express different pathways may be used in prescribing therapy. The methods and systems of the invention generally directed at digital microscopy are adapted to replace visual observation and image processing as an important aid to pathologists and researchers.

One or more of the embodiments of the systems and methods described uses TMAs comprising tissue from colon cancer patients and applies fluorescent based immunohistochemistry to identify the cell membrane (using pan-cadherin antibody), cMet (extracellular domain), and cell nuclei (using DAPI). A mask of the stromal region is generated, and using curvature and geometry based segmentation, the membrane and nuclear regions of the tumor region are demarcated. The cytoplasm is generally defined as the area between the membrane and nucleus or within the membrane space. Probability distributions of cMet within the membrane, nuclei or cytoplasm are determined, and an automated scoring algorithm that assigns cMet scores for each compartment is generated. Thresholds associated with a significant difference in survival time can also be determined using these systems and methods.

An example embodiment of the system for analyzing tissue samples, generally comprises: a storage device for at least temporarily storing one or more images of one or more cells, wherein at least one of the images is indicative of one or more channels comprising a receptor tyrosine kinase (RTK); and a processing device that determines an extent to which one or more of the RTKs may have translocated from at least one subcellular region to another subcellular region of one or more of the cells; and generates a score based at least in part on the RTK translocation, wherein at least one of the RTKs may be cMet. One or more of the subcellular regions may be at least a portion of a membrane, cytoplasm or nucleus. The score may used for a variety of purposes including, but not limited to, determining the location of the RTKs and their activation status in cancer and non-cancer tissues. For example, the score and location information may be used indicate whether the tissue comprises cancerous tissue such as, but not limited to, epithelial cancers such as, but again not limited to, breast cancer, colon cancer and melanoma. The scores and information may also be used to predict response to RTK-related therapies and research applications such as, but not limited to, drug discovery and cellular mechanisms of disease The images may reflect channels that are enhanced using one or more morphological stains and/or one or more biomarkers. The subcellular regions may comprise, but are limited to, the membrane, the cytoplasm, the stroma, epithelial nuclei, and stromal nuclei.

In one of the example embodiments, the processor determines the extent to which one or more of the RTKs has translocated at least in part by segmenting at least one of the images into one or more subcellular regions, and determining one or more metrics of translocation of at least one of the RTKs in a membrane region and in a cytoplasm region. In this embodiment, the processor may be configured to segment the images at least in part based on a probability map of a plurality of pixels making up the images; wherein the probability map reflects the likelihood that one or more of the pixels belongs to one or more of the subcellular regions. As noted, the subcellular regions may comprise, but are limited to, the membrane, the cytoplasm, the stroma, epithelial nuclei, and stromal nuclei.

In one of the example methods described, for analyzing tissue samples, the steps generally comprise: providing one or more images of one or more cells, wherein at least one of the images is indicative of one or more channels comprising a receptor tyrosine kinase (RTK); and determining an extent to which one or more of the RTKs may have translocated from at least one subcellular region to another subcellular region of one or more of the cells. The method may also generate a score based at least in part on the RTK translocation, wherein the score may indicate activation status, disease status and prognosis. The RTK may be, but is not necessarily limited to, cMet, and the subcellular regions may comprise, but are not limited to, the membrane, the cytoplasm, the stroma, epithelial nuclei, and stromal nuclei.

One of the example methods may determine the extent of translocation in part by segmenting at least one of the images into one or more subcellular regions; and may further comprise the step of determining a metric of translocation of at least one of the RTKs in a membrane region and in a cytoplasm region. The step of segmenting may, at least in part, use a probability map of a plurality of pixels making up the images, wherein the probability map reflects the likelihood that one or more of the pixels belongs to one or more of the subcellular regions.

### DRAWINGS

These and other features, aspects, and advantages of the present invention will become better understood when the following detailed description is read with reference to the accompanying drawings in which like characters represent like parts throughout the drawings, wherein:

FIG. 1 shows data and results for DAPI and IGg normalized cMet knockdown vs control cells used in one of the example embodiments of the systems and methods of the invention.

FIG. 2A shows the measured intensities of an embodiment of a multiple channel image using a plurality of markers showing the nuclei as stained with DAPI, the membrane as stained with pan-cadherin, and the target protein cMet.

FIG. 2B shows a probability map for the subcellar nuclear regions shown in FIG. 2A.

FIG. 2C shows a probability map for the subcellular membrane regions shown in FIG. 2A.

FIG. 2D shows a probability map for the several subcellular regions, the membrane, epithelial nuclei, stromal nuclei, and cytoplasm.

FIG. 2E shows the probability distribution function for one of the embodiments that, in this example uses cMet as the target protein, is used to determine the weighted distribution of the target protein.

FIG. 3 shows the distribution of a score overall and by the stages of a disease, as used in one of the embodiments that uses an MCT score and reflects a disease that exhibits a plurality of stages.

FIG. 4 shows four graphs of survival curves for all patients from an example of an embodiment described, the first of which reflects all three stages and the other three of which reflect stage 1-3 patients subdivided into high and low risk groups based on MCT score thresholds.

FIG. 5 is a schematic diagram of an embodiment of the automated system for carrying out the methods.

### DETAILED DESCRIPTION

One example member of the RTK family is cMet, also known as Met, hepatocyte growth factor or scatter factor receptor. CMet is a heterodimer with an extracellular domain (the alpha subunit), and a beta subunit, which is comprised of a transmembrane domain, juxtamembrane and kinase domain. The juxtamembrane domain has an inhibitory function for cMet activation, where phosphorylation of the serine residue (S985) or tyrosine residue (Y1003) results in ubiquitinization, endocytosis and degradation of cMet. The C-terminal tail contains a docking site responsible for recruitment of downstream signaling molecules. Under normal physiological conditions in adults it contributes to the maintenance of normal organ architecture and tissue patterning. In the activated state it has multiple roles in complex and interactive networks, including several signaling pathways associated with cell motility, size, shape, proliferation and survival. It also interacts with receptors of other families involved in cancer progression (including but not limited to plexins, EGF receptor family, Fas, integrin alpha 6 beta 4 and CD44).

Generally, the tissue used in the methods and systems, is fixed or otherwise provided on a substrate such as, but not limited to, a TMA, a slide, a well, or a grid. The tissue is labeled with molecular biomarkers, and imaged through a fluorescent microscope. Then the tissue is re-labeled with one or more morphological stains and imaged again. The methods are not limited to a particular sequence of applying one or more stains, nor are the methods limited to two images and can be adapted to co-register more than two images as needed. The images are overlaid using both hardware and software registration techniques, and the information is merged, whereby one of the technical effects is to co-register or otherwise produce multi-channel images.

Fluorescent-based immunohistochemistry is used in some of the example embodiments to identify cellular regions such as the cell membrane, cytoplasm and nuclei. Generally, a mask of the stromal region is generated, and using curvature and geometry based segmentation, the membrane and nuclear regions of a given tumor region are demarcated. The cytoplasm is designated as the area between the membrane and nucleus or within the membrane space. The probability distribution of cMet within the membrane, nuclei or cytoplasm, is determined, and a cMet score is generated, for each compartment. Thresholds associated with a significant difference in survival time may also be determined.

Any number and type of morphological markers may be used in the methods including, but not limited to, the following:

### Keratin: marker for epithelial cells

### Pan-cadherin: marker for the cell membrane

Smooth muscle actin: marker for muscle

### DAPI: marker for the nucleus

### Hematoxylin marker for DNA (blue stain)

### Eosin: marker for cytoplasm depends on pH (red stain).

### Some of these morphological markers can be imaged using a bright field microscope, and some with a fluorescent microscope.

The methods and systems of the invention may be used to personalized therapy and enhance disease research and cancer drug development. One example of the systems and methods uses pan-cadherin as a morphological stain for the cell membrane and cMet receptor tyrosine kinase as the biomarker.

### EXAMPLE

Beginning with TMAs of colon cancer (YTMA8 from Yale Tissue Microarray Facility), images corresponding to 583 patients were obtained. The median follow-up for these patients was 4.5 years, with 34% of the cases having more than 10 years of follow-up and 24% having more than 15 years of follow-up. Of the 583 patients, 485 (83%) were reported to have died and 98 (17%) patients were alive as of their last follow-up. However, only 264 patients (45%) of patients were considered to have died as a result of the disease. The overall median survival for this group of patients was 4.5 years, with 17% surviving as of last update of the data. Treatment information was not available for any of the subjects. Of the 583 patients, 437 (73%) had the primary tumor site as the colon, while the remaining 146 (27%) of cases had the primary tumor site as the rectum or other sites. All stages of colon cancer were represented in this cohort: 122 (21%) were stage I patients, 145 (25%) were stage II, 224 (38%) were stage III, and 59 (10%) were stage IV patients. Ninety percent of the cases were adenocarcinomas and 70% of the cases were well or moderately differentiated.

The tissue microarrays were constructed with histospots (0.6 mm in diameter) spaced 0.8 mm apart in a grid layout using a manual Tissue Microarrayer (Beecher Instruments, Silver Spring, Md.). The resulting tissue microarray blocks were cut to 5 μm sections with a microtome, the sections were placed on slides with an adhesive tape-transfer method (Instrumedics, Inc., Hackensack, N.J.), and were UV cross-linked for subsequent use in antibody optimization protocols.

A commercial antibody directed against the extracellular domain of cMet (clone DO-24, Upstate Biotechnology, Lake Placid, N.Y.) was optimized in this example for fluorescent immunohistochemistry in human tissue using formalin-fixed paraffin-embedded tissues (including colon, breast, stomach, prostate, kidney, muscle, liver, brain, skin and lymph) from the archives of the Yale University. Series of serial dilutions were evaluated on test tissue arrays, each containing 10-20 histospots, to identify the optimal concentration of DO-24 that provided a wide dynamic range of staining. In addition, a breast cancer cell line array called “MaxArray” (Zymed, San Francisco, cat# 75-3023) was used as a positive control for cMet staining and a “no primary” negative control slide was included. Cytoplasmic and membranous staining of cMet was observed in all tissue types and a DO-24 dilution of 1:2000 was determined to be optimal. Secondary antibody and TSA amplification detection used.

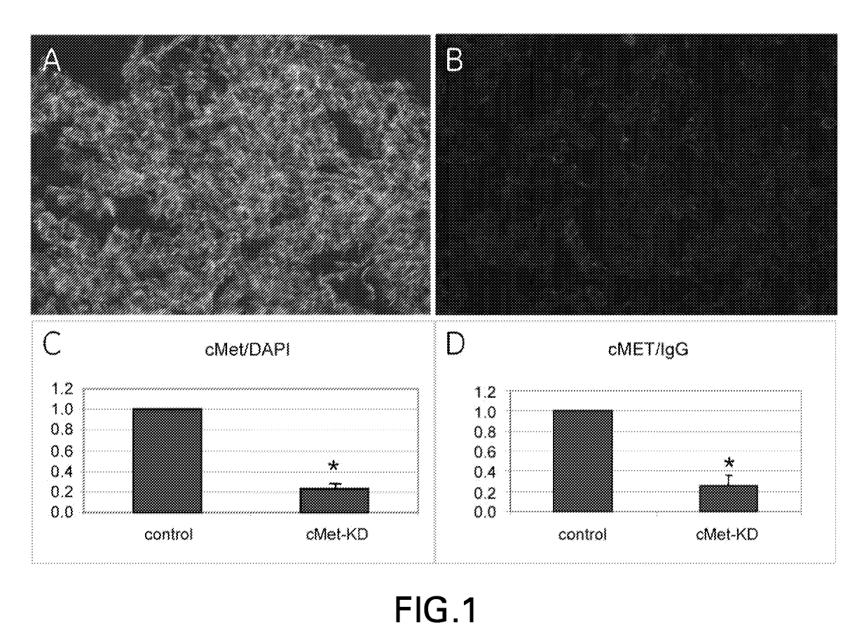

Specificity of the DO-24 antibody was evaluated using HeLa cells in which cMet protein was knocked down using RNA interference technology in comparison with control cells. The results for DAPI and IGg normalized knockdown vs control cells are shown in FIG. 1.

Conditions for siRNA transfection of HeLa cells, obtained from ATCC (Cat. CCL2) were previously established by evaluating combinations of concentrations of the positive transfection control BlockIT flurescein-labeled siRNA control (Invitrogen, Cat 2013) with Lipofectamine 2000 (Cat. 11667-027, Invitrogen) in 96 well plates using the InCell1000 (GE Healthcare). 20 nmole dry pellets of siRNA were resuspended in DEPC-treated water to create siRNAs in 20 mM Tris-HCL, pH 8, 20 mM NaCL, 1 mM EDTA. Of three independent RNA duplexes targeting cMET (METHSS106477, METHSS106478, METHSS106479, Invitrogen), METHSS106479 was found to give optimal knockdown, as determined by Western analysis using an anti-CMet antibody (C-12, Santa Cruz Biotechnology, Santa Cruz, Calif., Cat. Sc-10) to detect protein levels in RIPA buffer cell extracts.

Prior to transfection, cells were seeded in 100 cm² plates at a seeding density of 1.03×10⁶ cells/plate in MEM media containing 10% FBS, without antibiotics. The day of transfection, Lipofectamine 2000 (Cat. 11667-027, Invitrogen) was mixed with OptiMem media (Invitrogen) and allowed to incubate at RT. Universal negative siRNA control (46-2091), BlockIt control, or siRNA (METHSS106479) targeting cMET was incubated with OptiMem separately for 15 min. The diluted siRNA and Lipofecatamine were combined and incubated at RT for 15 min, then diluted into fresh MEM media containing serum but lacking antibiotics and added to the cells. The final concentration of siRNA in the media was 25 pMol. Cells were fixed following 48 hr culture with 4% PFA and embedded in paraffin, as previously described (Dolled-Filhart, McCabe et al. 2006). Five micron sections from each paraffin block were immunostained with DO-24 antibody at 1:2000, as described for the IHC of TMAs, except that the second blocking step used 10% donkey serum. Pan-cadherin was detected using anti-pan-cadherin at 1:100, 37 C, 1 hr (LabVision, Cat. RB9036)) with Cy3-conjugated AffiniPure F(ab)₂ fragment Donkey anti-rabbit IgG (Jackson ImmunoResearch, Cat. 711-166-152). Nuclei were stained with DAPI. Images were captured with a Zeiss ZI imager, average pixel intensity was measured, and background was subtracted and cMet values were normalized to DAPI. Statistical significance was determined using an unpaired t-test.

The TMAs were deparaffinized first by heating at 60° C., then by two xylene rinses followed by two rinses with 100% ethanol and a rinse in water. Antigen retrieval was performed in a Tris-EDTA buffer at a pH of 9.0 in the PT Module device (LabVision). After rinsing briefly in 1× Tris-buffered saline (TBS), a 30-minute incubation with 2.5% hydrogen peroxide/methanol block was used to block endogenous peroxidases, followed by incubation with 10% goat serum (Gibco, cat#16210-064) for one hour at room temperature. The anti-cMet antibody, DO-24, was incubated overnight at 4° C. at a dilution of 1:2000. The anti-cMet antibody was detected with Envision anti-mouse labeled polymer HRP (DAKO, product # K4001), followed by Cy5 tyramide (1:50, Perkin Elmer, product #SAT705A). All slides were then incubated overnight at 4° C. in a cocktail of a rabbit anti-pan-cadherin antibody (Abcam, 1:1000, product #Ab6529-200) for membrane identification and a monoclonal mouse anti-cytokeratin antibody (clone AE1/AE3, DAKO, 1:200, product #M3515). Pan-cadherin was detected by incubation with biotin goat anti-rabbit (Jackson ImmunoResearch, product #111-065-144, 1:200) for 60 minutes, followed by Cy-2 conjugated streptavidin (Jackson ImmunoResearch, product #016-220-084, 1:200) for 30 minutes. Slides were mounted using Prolong Gold Anti fade w/DAPI mounting gel (Invitrogen/Molecular Probes, product #P36931). Images were captured using a PM-2000™ microscopy platform (HistoRx, New Haven, Conn.) at 20× magnification with an exposure time for the Cy5 channel of 400 ms.

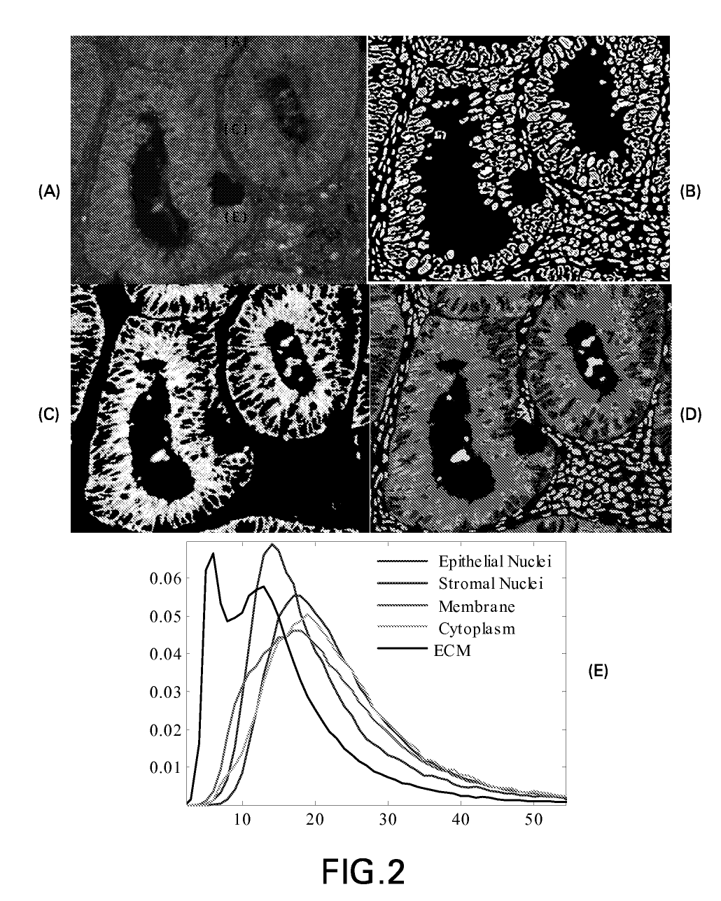

Once the images are obtained, the multiple channel digital images are partitioned into multiple regions (segments/compartments) to quantify one or more biomarkers, such as cMet. However, the quantification can be accomplished without determining definite decisions for each pixel, but rather by computing the likelihood that a given pixel belongs to a cellular region. For example, instead of identifying membrane pixels, the likelihood of a pixel being a membrane can be computed, which is essentially the probability of a pixel being a membrane. Probability maps of these pixel regions are computed using the intensity and geometry information provided by each channel. The preferred methods for created these probability maps are disclosed in U.S. application Ser. No. 11/680,063, entitled Automated Segmentation of Image Structures, which is incorporated by reference. FIG. 2A shows the measured intensities of a multiple channel image using a plurality of markers showing the nuclei as stained with DAPI, the membrane as stained with pan-cadherin, and the target protein cMet. The probability maps computed for the nuclei and membrane are shown in FIGS. 2B and 2C, respectively. The brightness of these images represents the probability value; white representing the probability value of one, black representing the probability value of zero, and any shade of gray being proportional with the probability value. A definite decision for each pixel may be determined by thresholding the probability maps. Such decisions are used in one or more of the embodiments to separate the epithelial nuclei from the stromal nuclei, and to detect the cytoplasm. The cytoplasm is also represented as a probability map of ones and zeros. FIG. 2D shows the computed different regions; membrane, epithelial nuclei, stromal nuclei, and cytoplasm. Regions are excluded in this embodiment of the quantification step. Both the background and the extra cellular matrix are shown as black.

Translocation of a target protein between different regions is quantified in one or more of the embodiments using probability mapping. The distribution of cMet in each of the regions was represented by a probability distribution function (PDF). FIG. 2E shows the PDF of the target cMet on each of the regions. For example the PDF of cMet on the membrane is the weighted empirical distribution of the cMet, where the membrane probability map determines the weights. The mean and the standard deviation of cMet distribution are denoted in this example on each of the regions as μ_{R}, and σ_{R}, respectively, where R can be any of the nuclei, membrane, cytoplasm or non-epithelial extra cellular matrix (ECM) regions. ECM in this example is defined as all the non-background pixels not classified as nuclei, membrane or cytoplasm. In this example, the translocation score is the normalized mean difference between the cMet distributions on different regions. For example the membrane to cytoplasm translocation (MCT) score is,

$$\frac{{μ}_{Membrane} - {μ}_{Cytoplasm}}{\sqrt{{σ}_{Membrane}^{2} + {σ}_{Cytoplasm}^{2}}} . \qquad ( 4 )$$

where the mean and the standard deviation of any region, R, are defined using the cMet PDF, f_{C}^{R}(c), on that region;

$$\begin{matrix}{μ}_{R} = {\sum }_{c} {cf}_{C}^{R} ( c ) & ( 5 ) \\ {σ}_{R}^{2} = {\sum }_{c} {( c - {μ}_{R} )}^{2} {f}_{C}^{R} ( c ) . & ( 6 )\end{matrix}$$

In this example, five regions were detected (epithelial nuclei, cytoplasm, membrane, extra cellular matrix (ECM) and ten different translocation scores, (5 choose 2), were generated. The translocation score is defined in this embodiment as the normalized mean difference between the corresponding PDFs. All translocation scores are then related with clinical outcome, and tested for statistical significance to explore the association with the life expectancy.

A robust and cross-validated method, described below, is used in this embodiment to define an optimal threshold for each score that separates the patients into two groups having a maximum difference in survival. The two groups defined by the threshold are then considered relative to each other as the low risk and high risk groups. For example, a threshold may be used to minimize the log rank test p-value between the two groups, with adjustments for multiple testing as the threshold is determined by repeated testing at each possible cut point. However, to make the threshold robust to variation across data sets, the following cross-validated steps may be used:

a. 2-fold Cross-validation: The data is divided into training and test data sets, with 50% of the cases (both censored and uncensored) being selected as training and the remaining as test;

b. Threshold Selection: For each training data set, an optimal threshold is selected by the minimizing the log rank test p-value and an adjusted p-value (for multiple testing) is also computed;

c. Cross-validation of Threshold: The optimal threshold is applied to the corresponding test data set and the log rank p-value for comparing the two groups is computed;

d. Computing a robust threshold: The above three steps are repeated 500 times and the median of thresholds, hazard ratios and p-values from the 500 cross-validation runs is used. The final robust threshold is this median threshold.

Test of association between risk category and clinical/pathological stage variables are performed using chi-square test of association in a contingency table. A Kaplan-Meier method may be used to estimate the median survival and survival rates in each risk group and Cox regression used to assess the prognostic value of the risk category in a multivariate analysis. In multivariate analysis, a stepwise selection procedure (SAS v9.0) is used to select the variables that are independent prognostic factors.

With median age of 68 years and a follow-up of more than 10 years on 34% of cases, it was expected that significant proportion of deaths could be unrelated to disease. A careful analysis of the cause of death was undertaken to rule out death from causes unrelated to the cancer. Analyses presented are primarily therefore based on death due to disease.

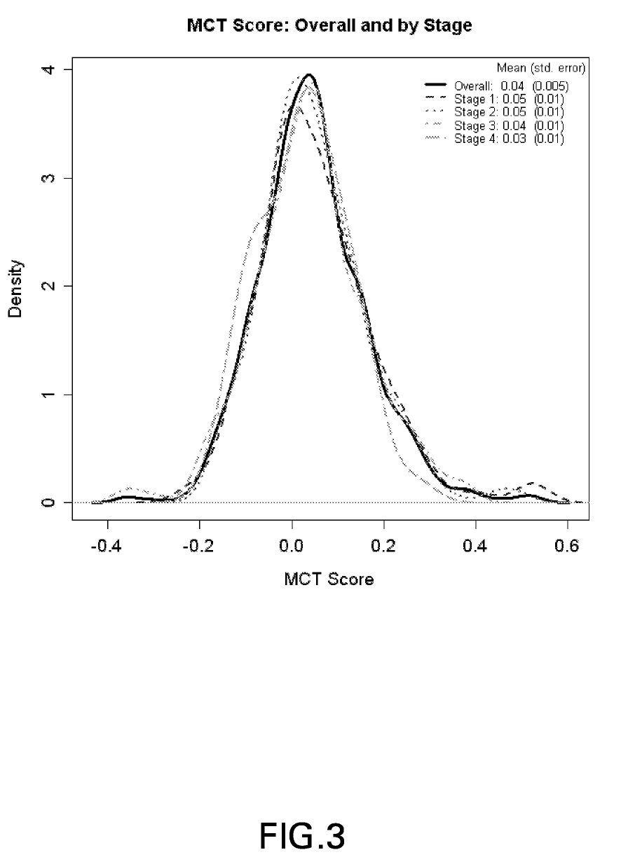

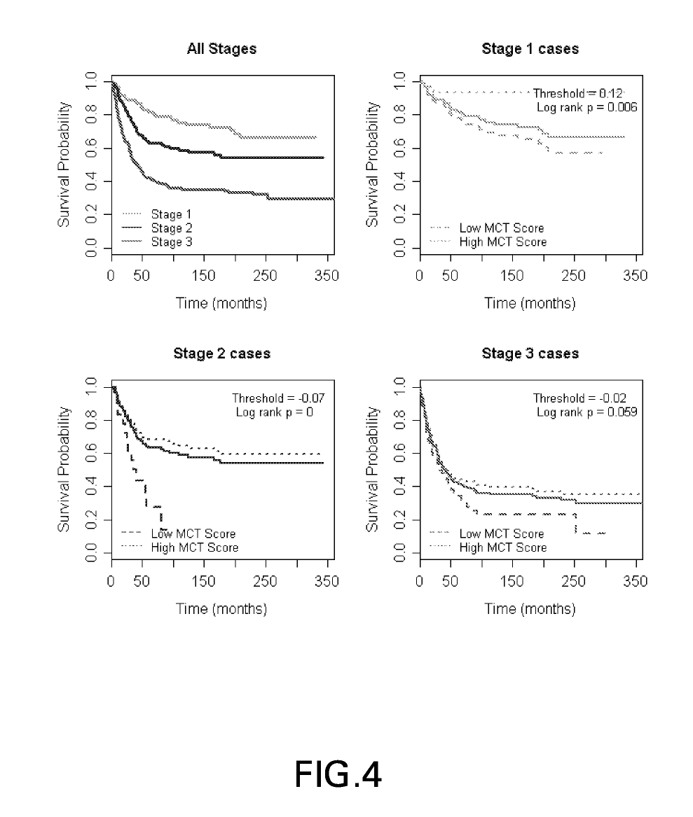

Of the possible combinations of translocation scores from the subcellular compartments of nuclei, cytoplasm, and membrane generated in this embodiment, the membrane cytoplasm translocation score (MCT score) is the preferred predictor of survival (training and test-set p-values in the cross-validation study<0.1). FIG. 3 shows the distribution of the MCT score overall and by stage. The mean MCT score for all 583 cases in this embodiment is 0.04 (sd=0.12) and there is no difference (p-value of anova F-test=0.4) in the distribution of the MCT score by stage. FIG. 4A shows the survival curves for all patients, while FIGS. 4B-D show the same survival curves for stages 1-3 patients, respectively, subdivided into high and low risk groups based on the MCT score thresholds.

For this example, 500 random cross-validation runs were used to select a robust and validated threshold for the membrane, cytoplasm and MCT scores. For the MCT score, thresholds were identified that significantly differentiated patients (all, stage I and stage II) into higher risk and lower risk groups. For example, higher risk patients with a shorter survival time had low MCT scores and lower risk patients with a longer survival time had high MCT scores. In this example, for all patients combined, the threshold MCT score was identified as −0.07, which means that a score of −0.07 and lower indicated a poor prognosis overall. For stage I patients, the threshold was 0.12 and for stage III patients, the threshold was −0.07. The results of the 500 cross-validation runs are summarized in Table 1 for deaths due to the disease as the endpoint, showing the median split points, hazard ratios and p-values from the 500 cross-validation runs. In Table 1, the membrane score and the cytoplasm score reflect subtraction of the ECM from the membrane and subtraction of the ECM from the cytoplasm, respectively.

TABLE 1
Results of two-fold cross-validation of threshold selection on
the membrane, cytoplasm and membrane cytoplasm translocation
scores with death due to disease as the endpoint.

| | Overall | Stage I | Stage II | Stage III | Stage IV |
|---|---|---|---|---|---|
| Membrane Score | | | | | |
| Optimal Split Point | 0.78 | 0.63 | 0.835 | 0.845 | 0.53 |
| Median survival (months) in Low | 192; 186 | Median | Median | 37; 40 | Median |
| MCT Group; Median survival | | survival not | survival not | | survival not |
| (months) in High MCT group | | reached | reached | | reached |
| Hazard Ratio of High vs Low MCT | 0.79 | 2.305 | 1.81 | 0.63 | 3.46 |
| Score (training data) | | | | | |
| p-value (training data) | 0.09 | 0.09 | 0.14 | 0.08 | 0.09 |
| Adjusted p-value (training data) | 0.70 | 0.70 | 0.82 | 0.62 | 0.58 |
| p-value (test data) | 0.59 | 0.66 | 0.44 | 0.52 | 0.43 |
| Cytoplasm Score | | | | | |
| Optimal Split Point | 0.65 | 0.77 | 0.93 | 0.64 | 0.6 |
| Median survival (months) in Low | 202; 176 | Median | Inf; 156.5 | 42; 36 | Median |
| MCT Group; Median survival | | survival not | | | survival not |
| (months) in High MCT group | | reached | | | reached |
| Hazard Ratio of High vs Low MCT | 1.49 | 2.68 | 2.26 | 0.7 | 2.4 |
| Score (training data) | | | | | |
| p-value (training data) | 0.06 | 0.09 | 0.04 | 0.11 | 0.12 |
| Adjusted p-value (training data) | 0.52 | 0.69 | 0.44 | 0.75 | 0.76 |
| p-value (test data) | 0.69 | 0.54 | 0.34 | 0.53 | 0.56 |
| MCT Score | | | | | |
| Optimal Split Point | −0.07 | 0.12 | −0.07 | −0.02 | 0.01 |
| Median survival (months) in Low | 57; 220 | Median | Inf; 171 | 41; 37 | Median |
| MCT Group; Median survival | | survival not | | | survival not |
| (months) in High MCT group | | reached | | | reached |
| Hazard Ratio of High vs Low MCT | 0.56 | 0 | 0.28 | 0.58 | 2.56 |
| Score | | | | | |
| p-value (training data) | 0.007 | 0.02 | 0.002 | 0.04 | 0.05 |
| Adjusted p-value (training data) | 0.17 | 0.20 | 0.15 | 0.51 | 0.57 |
| p-value (test data) | 0.08 | 0.07 | 0.07 | 0.45 | 0.6 |

For the MCT score, the median hazard ratio between the high MCT score group and the low MCT score group is 0.56 in all patients (median log rank p-value=0.08 in the test data). The MCT score also has a test set log rank p-value of 0.07 in stage I and stage II patients. The median of the optimal split points is −0.07 in all patients, 0.12 in stage I patients and −0.07 in stage II patients. This threshold is applied in this example to the entire data set and the patients are categorized as low risk (MCT>=−0.07) and high risk (MCT<−0.07). Of the 583 cases, 93 cases (16%) are categorized as high risk (MCT score below threshold) and the remaining 490 cases are low risk (MCT score above threshold). In this example, the percentages of cases that fell into the high-risk category are: 14% in Stage I, 14% in Stage II, 17% in Stage III, and 22% in Stage IV. The clinical and pathological data are then correlated with the expression of cMet. The associative correlations between clinical and pathological variables, such as but not limited to, age, stage of disease at diagnosis, pathological stage, histology and histology grade, and MCT score group are summarized in Table 2.

TABLE 2
Demographic, Clinical and Pathological Data for high and low MCT Score Groups

| | | All Cases | | | | Stage I Cases | | | | Stage II Cases | | | |
|---|---|---|---|---|---|---|---|---|---|---|---|---|---|
| | | Threshold = −0.07 | | | | Threshold = 0.12 | | | | Threshold = −0.07 | | | |
| | | | Low MCT | High MCT | | | Low MCT | High MCT | | | Low MCT | High MCT | |
| | | | Score: | Score: | | | Score: | Score: | | | Score: | Score: | |
| | | N | N (%) | N (%) | P | N | N (%) | N (%) | P | N | N (%) | N (%) | P |
| Overall | | 583 | 93 | 490 | | | | | | 145 | 20 | 125 | |
| Age | <70 | 313 | 48 (15%) | 265 (85%) | 0.66 | 69 | 52 (75%) | 17 (25%) | 0.82 | 68 | 7 (10%) | 61 (90%) | 0.25 |
| | ≧70 | 270 | 45 (17%) | 225 (83%) | | 53 | 39 (74%) | 14 (26%) | | 77 | 13 (17%) | 64 (83%) | |
| Tumor Site | Colon | 437 | 74 (17%) | 363 (83%) | 0.26 | 82 | 64 (78%) | 18 (22%) | 0.21 | 117 | 19 (16%) | 98 (84%) | 0.08 |
| | Rectum/ | 146 | 19 (13%) | 127 (87%) | | 40 | 27 (68%) | 13 (33%) | | 28 | 1 (4%) | 27 (96%) | |
| | Other | | | | | | | | | | | | |
| Stage at | Distant/ | 82 | 13 (16%) | 69 (84%) | 0.98 | 8 | 6 (75%) | 2 (25%) | 0.23 | 16 | 2 (12%) | 14 (88%) | 0.78 |
| Diagnosis | Unknown/ | | | | | | | | | | | | |
| | Other | | | | | | | | | | | | |
| | Localized | 245 | 40 (16%) | 205 (84%) | | 106 | 77 (73%) | 29 (27%) | | 55 | 9 (16%) | 46 (84%) | |
| | Reg | 256 | 40 (16%) | 216 (84%) | | 8 | 8 (100%) | 0 (0%) | | 74 | 9 (12%) | 65 (88%) | |
| Histology | Adenocar- | 524 | 79 (15%) | 445 (85%) | 0.09 | 114 | 84 (74%) | 30 (26%) | 0.39 | 132 | 16 (12%) | 116 (88%) | 0.06 |
| | cinoma | | | | | | | | | | | | |
| | Other | 59 | 14 (24%) | 45 (76%) | | 8 | 7 (88%) | 1 (13%) | | 13 | 4 (31%) | 9 (69%) | |
| Histology | Mod Diff | 229 | 37 (16%) | 192 (84%) | 0.48 | 50 | 39 (78%) | 11 (22%) | 0.4 | 61 | 7 (11%) | 54 (89%) | 0.32 |
| Grade | Poorly | 57 | 6 (11%) | 51 (89%) | | 5 | 5 (100%) | 0 (0%) | | 12 | 0 (0%) | 12 (100%) | |
| | Diff | | | | | | | | | | | | |
| | Unknown/ | 118 | 23 (19%) | 95 (81%) | | 23 | 17 (74%) | 6 (26%) | | 20 | 3 (15%) | 17 (85%) | |
| | Undiff/N | | | | | | | | | | | | |
| | ot graded | | | | | | | | | | | | |
| | Well Diff | 179 | 27 (15%) | 152 (85%) | | 44 | 30 (68%) | 14 (32%) | | 52 | 10 (19%) | 42 (81%) | |
| T Stage | T0/T1 | 21 | 1 (5%) | 20 (95%) | 0.33 | | | | | | | | |
| | T2 | 178 | 31 (17%) | 147 (83%) | | | | | | | | | |
| | T3/T4/ | 384 | 61 (16%) | 323 (84%) | | | | | | | | | |
| | Unknown | | | | | | | | | | | | |
| N Stage | N0 | 277 | 38 (14%) | 239 (86%) | 0.27 | | | | | | | | |
| | N1 | 147 | 29 (20%) | 118 (80%) | | | | | | | | | |
| | N2/ | 159 | 26 (16%) | 133 (84%) | | | | | | | | | |
| | Unknown/ | | | | | | | | | | | | |
| | Nx | | | | | | | | | | | | |
| TNM Stage | I | 122 | 17 (14%) | 105 (86%) | 0.45 | | | | | | | | |
| | II | 145 | 20 (14%) | 125 (86%) | | | | | | | | | |
| | III | 224 | 38 (17%) | 186 (83%) | | | | | | | | | |

For all 583 cases in this example, no association exists between MCT score-based risk groups and any of the clinical-pathological variables. While, as expected, the proportion of high-risk (low MCT score) patients increased from age ≦70 to age >70 (from 15% to 17%), this association is not significant. As such, the MCT score group and age are not confounded and indicate independent effects on survival. Similarly, although the proportion of high-risk patients (low MCT score) increased in this example with the stages (e.g. 14% in stage 1 to 22% in stage IV), this trend is not significant. In addition, only a marginal association exists in the example group with histology (e.g. a larger proportion (24%) of patients with non-adenocarcinomas being in the low MCT score/high risk category than those with adenocarcinomas (15%)).

Univariate and multivariate survival analyses were performed. Using a univariate survival analysis, the median survival in the low risk group (low MCT score) was 55 months with a 95% confidence interval of [41, 122] months while the high-risk group (high MCT score) had a median survival of 228 months with a 95% confidence interval of [167, inf* indicating median was not attained] months. The low MCT score group 5-year survival rate was 48% with a 95% confidence interval of [38%, 60%] and the high MCT score group 5-year survival rate was 62% with a 95% confidence interval of [57%, 66%].

The results of univariate Cox regression on membrane, cytoplasm, MCT scores and other clinical and pathological variables are shown in Table 3.

TABLE 3
Univariate analysis

| | All Cases | | | | Stage II Cases | |
|---|---|---|---|---|---|---|
| | Threshold = −0.07 | | Stage I Cases | | Threshold = −0.07 | |
| | | Log | Threshold = 0.12 | | Hazard | |
| | Hazard Ratio | Rank P | Hazard Ratio | Log Rank P | Ratio | Log Rank P |
| *Membrane Score: Low vs High | 0.83 | 0.13 | 0.7 | 0.32 | 1.06 | 0.82 |
| *Cytoplasm Score: Low vs High | 0.86 | 0.21 | 0.9 | 0.75 | 2.07 | 0.008 |
| *MCT Score: Low vs High | 0.67 | 0.009 | 0.16 | 0.006 | 0.34 | 0.0005 |
| Age: >= 70 vs <70 | 1.27 | 0.05 | 0.63 | 0.25 | 1.43 | 0.19 |
| Tumor Site: Rectu vs Colon | 1.22 | 0.14 | 1.83 | 0.09 | 1.52 | 0.17 |
| Stage at Diagnosis: Localized vs Not | 0.08 | <0.0001 | 0.02 | <0.0001 | 0.19 | <0.0001 |
| Stage at Diagnosis: Regional vs Not | 0.21 | <0.0001 | 0.1 | 0.001 | 0.25 | <0.0001 |
| Histology: Other vs Adenocarcinoma | 1.05 | 0.81 | 0.39 | 0.35 | 1.5 | 0.35 |
| Histology Grade: Poorly Diff vs Not | 1.35 | 0.14 | 1.67 | 0.5 | 0.17 | 0.08 |
| Histology Grade: Unknown/ | 0.85 | 0.34 | 0.46 | 0.22 | 0.97 | 0.95 |
| Undiff/Not graded vs Not | | | | | | |
| Histology Grade: Well Diff vs Not | 0.81 | 0.17 | 1.2 | 0.64 | 1.02 | 0.96 |
| T Stage: T2 vs not T2 | 1.22 | 0.67 | | | | |
| T Stage: T3/T4/Unknown vs Not | 2.43 | 0.05 | | | | |
| T3/T4/Unknown | | | | | | |
| N Stage: N1 vs Not N1 | 2.15 | <0.0001 | | | | |
| N Stage: N2/Nx/Unknown vs Not | 2.58 | <0.0001 | | | | |
| TNM Stage: II vs not II | 1.76 | 0.012 | | | | |
| TNM Stage: III vs Not III | 3.53 | <0.0001 | | | | |
| TNM Stage: IV vs Not IV | 2.02 | 0.0084 | | | | |

*Unadjusted p-value when threshold is applied to entire data set.

The MCT score group as defined by the threshold was a significant predictor of survival with hazard ratio of 0.67 (p=0.01) for survival (death due to disease only). The prognostic value of MCT score is particularly pronounced in stage I and II cases, for which the MCT score risk category in this example has a hazard ratio of and 0.34 (p=0.0005) and survival (death due to disease only) respectively. The other significant predictors of survival from death due to disease are age, stage of diagnosis, N Stage, T Stage and overall TNM Stage, as shown in Table 4. For the stage I and II cases in this example, stage at diagnosis (p<0.0001) is the only other significant predictor of survival from death due to disease in univariate Cox regression, though age was also a significant predictor when overall survival is considered.

Variables considered significant in univariate analyses are included in this example of multivariate analyses and stepwise selection is used to select those that are significant predictors of survival in multivariate analysis. The results of the multivariate Cox regression with stepwise selection are shown in Table 4. The MCT score is selected as a significant predictor even with stepwise selection indicating its significance as an independent prognostic factor for both survival and survival from death due to disease. In this example, among the Stage I patients, MCT score is the only significant predictor selected in multivariate Cox regression with hazard ratio=0.16, p<0.001). Among the stage II patients, MCT score is also the only significant predictor selected with hazard ratio=0.34, p<0.001).

As shown in Table 4, for all 583 cases, disease location at diagnosis, N Stage, TNM stage, MCT score and age are to significant to varying degrees. When the multivariate Cox regression is stratified by stage at diagnosis (not shown in Table 5), the only significant predictors are age (hazard ratio of ≦70 vs >70=1.5, p=0.001) and MCT score (hazard ratio for high vs low score group=0.68, p=0.012).

TABLE 4
Multivariate Analysis:

| | All Stages | | Stage I | | Stage II | |
|---|---|---|---|---|---|---|
| | Hazard | | Hazard | | Hazard | |
| | Ratio | P | Ratio | P | Ratio | P |
| MCT: High vs | 0.7 | 0.03 | 0.16 | 0.006 | 0.34 | 0.001 |
| Low | | | | | | |
| Age Group | 1.5 | 0.002 | | | | |
| Stage at | 0.21 | <0.0001 | 0.08 | 0.0002 | | |
| Diagnosis: | | | | | | |
| Regional | | | | | | |
| vs not Regional | | | | | | |
| Stage at | 0.1 | <0.0001 | 0.02 | <0.0001 | | |
| Diagnosis: | | | | | | |
| Localized vs Not | | | | | | |
| Localized | | | | | | |
| N Stage: N2 vs | 1.55 | 0.0025 | | | | |
| Not N2 | | | | | | |
| TNM Stage: | 1.51 | 0.003 | | | | |
| Stage III vs | | | | | | |
| not Stage III | | | | | | |

The methods and systems comprise automated scoring methods that employ curvature-based segmentation and assign probability distributions of a biomarker within a given area. In one or more of the embodiments of the methods, cMet cytoplasm to membrane distribution is used a predictor of outcome in: overall population; and in a more specific embodiment in stage I and II colon cancer patients.

One or more of the embodiments of the methods and systems effectively generates a score for cancer patients, and in more specific embodiments generates a score for colon cancer, referred to a membrane to cytoplasm translocation score (MCT score), that is a significant predictor of disease related survival and, in more specific embodiments, a predictor of overall survival in stage I and II patients.

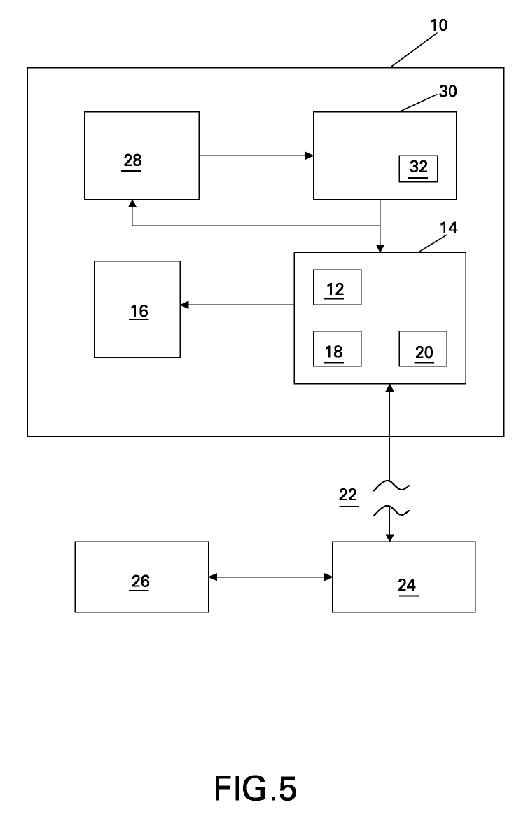

The automated system 10 (FIG. 5) for carrying out the methods generally comprises: a storage device 12 for at least temporarily storing one or more images of one or more cells, wherein the images comprise a plurality of channels; and a processor 14 comprising, a means for determining an extent to which a biomarker may have translocated from at least one subcellular region to another subcellular region; and a means for generating a score corresponding to the extent of translocation. The score may indicate whether the patient's prognosis is poor and whether the tissue is cancerous and/or is metastasizing. The system may be adapted to determine scores directed at translocation that indicates the presence of cancer. The system may be further adapted to determine translocation indicative of a specific group of cancers such as epithelial cancer and more specifically colon cancer. However, the system is not necessarily limited to epithelial cancers or to colon cancer.

The means for determining the extent of the translocation may comprise one or more processing mechanisms such as one or more algorithms residing in a memory device associated with the processor or a sub processor, or an external processor that is capable of communicating with the processor.

The mean for determining the extent to which the biomarker has translocated may determine the translocation at least in part using one or more appropriate segmentation steps of the methods. For example, the system may segment at least one image comprising a biomarker channel and at least one image comprising a morphological channel into subcellular regions, and a metric of translocation of the biomarker in a membrane subcellular region and another subcellular region.

The storage device may comprise, but is not necessarily limited to, any suitable hard drive memory associated with the processor such as the ROM (read only memory), RAM (random access memory) or DRAM (dynamic random access memory) of a CPU (central processing unit), or any suitable disk drive memory device such as a DVD or CD, or a zip drive or memory card. The storage device may be remotely located from the processor or the means for displaying the images, and yet still be accessed through any suitable connection device or communications network including but not limited to local area networks, cable networks, satellite networks, and the Internet, regardless whether hard wired or wireless. The processor or CPU may comprise a microprocessor, microcontroller and a digital signal processor (DSP).

The storage device 12 and processor 14 may be incorporated as components of an analytical device such as an automated high-throughput system that stains and images the TMAs in one system and still further analyzes the images the generate a score. One of more of these steps may be configured into one system or embodied in one or more stand-alone systems. System 10 may further comprise a means for displaying 16 one or more of the images; an interactive viewer 18; a virtual microscope 20; and/or a means for transmitting 22 one or more of the images or any related data or analytical information over a communications network 24 to one or more remote locations 26.

The means for displaying 16 may comprise any suitable device capable of displaying a digital image such as, but not limited to, devices that incorporate an LCD or CRT. The means for transmitting 22 may comprise any suitable means for transmitting digital information over a communications network including but not limited to hardwired or wireless digital communications systems. The system may further comprise an automated device 28 for applying one or more of the stains and a digital imaging device 30 such as, but not limited to, an imaging microscope comprising an excitation source 32 and capable of capturing digital images of the TMAs. Such imaging devices are preferably capable of auto focusing and then maintaining and tracking the focus feature as needed throughout processing.

These multi-channel methods are not limited to morphological stains or fluorescent biomarkers or even to pathology. Any stain that enables some informative aspect or feature of a biological sample to be visualized so that it can be digitally imaged and processed would be suitable for these methods. Suitable stains include, but are not necessarily limited to, cytological or morphological stains, immunological stains such as immunohisto- and immunocyto-chemistry stains, cytogenetical stains, in situ hybridization stains, cytochemical stains, DNA and chromosome markers, and substrate binding assay stains. Other medical and bioscience applications can benefit from the extended multi-channels. These multi-channel methods provide a flexible framework in which markers can be imaged sequentially without being limited to optical, chemical, and biological interactions.

As noted, the methods and systems are suitable for any number of applications including, but not limited to, detecting and analyzing epithelial cancers such as, but not limited to, breast, colon and prostate cancers and melanoma.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for analyzing tissue samples, comprising,
a storage device for at least temporarily storing one or more images of one or more cells, wherein at least one of said images is indicative of one or more channels comprising a receptor tyrosine kinase (RTK); and
a processing device that determines an extent to which one or more of said RTKs may have translocated from at least one subcellular region to another subcellular region of one or more of said cells; and generates a score based at least in part on said RTK translocation.

2. The system of claim 1, wherein said score indicates whether said tissue comprises a diseased tissue

3. The system of claim 1, wherein at least one of said RTKs is cMet.

4. The system of claim 3, wherein one of said subcellular regions is at least a portion of a cellular membrane and one of said subcellular regions is at least a portion of cytoplasm.

5. The system of claim 4, wherein the tissue comprises colon cancer.

6. The system of claim 1, wherein one of said subcellular regions is at least a portion of a cellular membrane and one of said subcellular regions is at least a portion of cytoplasm.

7. The system of claim 2, wherein said tissue comprises an epithelial cancer.

8. The system of claim 7, wherein said epithelial cancer is from a group consisting of breast cancer, colon cancer and melanoma.

9. The system of claim 2, wherein said tissue comprises colon cancer.

10. The system of claim 1, wherein at least one of said images is indicative of a morphological stain.

11. The system of claim 10, wherein at least one of said morphological stains is pan-cadherin.

12. The system of claim 1, wherein at least one of said RTKs is cMet and at least one of said images is indicative of a morphological stain.

13. The system of claim 12, wherein said morphological stain is pan-cadherin.

14. The system of claim 12, wherein at least one of said subcellular regions is at least a portion of a membrane region.

15. The system of claim 14, wherein at least one of said subcellular regions is at least a portion of a cytoplasm region.

16. The system of claim 15, wherein at least one of said subcellular regions is at least a portion of a nuclear region.

17. The system of claim 1, wherein one or more of said subcellular regions is selected from a group consisting of at least a portion of: a membrane region, a cytoplasm region and a nuclear region.

18. The system of claim 1, wherein said processor determines said extent to which one or more of said RTKs has translocated at least in part by segmenting at least one of said images into one or more subcellular regions, and determining a metric of translocation of at least one of said RTKs in a membrane region and in a cytoplasm region.

19. The system of claim 18, wherein said processor segments said images at least in part based on a probability map of a plurality of pixels making up said images.

20. The system of claim 19, wherein said probability map reflects the likelihood that one or more of said pixels belongs to one or more of said subcellular regions.

21. The system of claim 1, wherein said processor determines said extent to which one or more of said RTKs has translocated at least in part by segmenting at least one of said images into subcellular regions, and determining a metric of translocation of at least one of said RTKs in at least a portion of a membrane region, a cytoplasm region and a nuclear region.

22. A method for analyzing tissue samples, comprising the steps of,
providing one or more images of one or more cells, wherein at least one of said images is indicative of one or more channels comprising a receptor tyrosine kinase (RTK); and
determining an extent to which one or more of said RTKs may have translocated from at least one subcellular region to another subcellular region of one or more of said cells.

23. The method of claim 22, further comprising the step of, generating a score based at least in part on said RTK translocation.

24. The method of claim 23, wherein said score indicates whether said tissue is diseased

25. The method of claim 23, wherein at least one of said RTKs is cMet.

26. The method of claim 25, wherein one of said subcellular regions is at least a portion of a cellular membrane and one of said subcellular regions is at least a portion of cytoplasm.

27. The method of claim 22, wherein at least one of said RTKs is cMet.

28. The method of claim 27, wherein the tissue comprises colon cancer.

29. The method of claim 22 wherein at least one of said images is indicative of a morphological stain.

30. The method of claim 29, wherein at least one of said morphological stains is pan-cadherin.

31. The method of claim 22, wherein at least one of said subcellular regions is at least a portion of a membrane region.

32. The method of claim 22, wherein at least one of said subcellular regions is at least a portion of a cytoplasm region.

33. The method of claim 22, wherein at least one of said subcellular regions is at least a portion of a nuclear region.

34. The method of claim 22 wherein said step of determining comprises segmenting at least one of said images into one or more subcellular regions.

35. The method of claim 34, wherein said step of determining further comprises the step of determining a metric of translocation at least one of said RTKs in a membrane region and in a cytoplasm region.

36. The method of claim 34, wherein step of segmenting at least in part uses a probability map of a plurality of pixels making up said images.

37. The method of claim 36, wherein said probability map reflects the likelihood that one or more of said pixels belongs to one or more of said subcellular regions.
